(19) United States
(12) Patent Application Publication
(10) Pub. No.: US 2024/0350096 A1
(43) Pub. Date: Oct. 24, 2024

(54) LOSS OF CONTROL DETECTION, ALERTS, AND/OR MANAGEMENT THEREOF

(71) Applicant: SynchNeuro, Inc., Philadelphia, PA (US)

(72) Inventor: Casey HALPERN, Philadelphia, PA (US)

(21) Appl. No.: 18/688,094

(22) PCT Filed: Aug. 31, 2022
(86) PCT No.: PCT/US2022/075755

§ 371 Date: Feb. 29, 2024

Related U.S. Application Data
(60) Provisional application No. 63/260,779, filed on Aug. 31, 2021.

(51) Int. Cl. A61B 5/00; A61B 5/145

(52) U.S. Cl. CPC A61B 5/7275; A61B 5/14532; A61B 5/747

(57) ABSTRACT

Methods that initiate an alert of an upcoming Loss of Control (“LOC”) event, and devices and systems that are configured to alert or initiate an alert about of an upcoming LOC event, wherein the method is adapted to receive as input sensed patient neural data or information that is indicative of sensed patient neural data, and detect an upcoming LOC event using at least the sensed patient neural data or the information indicative of the sensed patient neural data.

### CROSS REFERENCE TO RELATED APPLICATIONS

This application claims priority to U.S. Provisional Application 63/260,779, filed Aug. 31, 2021, the entire disclosure of which is incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference. PCT publication WO/2018064225 is incorporated by reference herein in its entirety for all purposes.

### BACKGROUND OF THE INVENTION

People with a disorder or condition exacerbated by poor impulse control and compulsivity cannot resist the urge to do something harmful to themselves or others. Loss of control (“LOC”) defined herein as an inability to control impulses or compulsions or any cravings or dangerous thoughts as they relate to substance abuse, alcohol use disorders and binge alcohol drinking, sex addiction or compulsive sexuality, kleptomania, pyromania, trichotillomania, panic disorder, Intermittent Explosive Disorder, compulsive behaviors including gambling, binge eating, night eating, loss of control eating as it relates to binge eating, obesity, and eating disorders, emotional or stress eating, compulsive eating, purge behaviors, suicidal ideation/planning/attempt, and other compulsive behaviors.

Systems and methods are needed that can provide information to the patient about an upcoming LOC event and help manage these events.

### SUMMARY OF THE DISCLOSURE

One aspect of this disclosure is a computer executable method adapted to initiate an alert of an upcoming Loss of Control (“LOC”) event.

In this aspect, the method may be adapted to receive as input sensed patient neural data or information that is indicative of sensed patient neural data, and detect an upcoming LOC event using at least the sensed data or the information indicative of the sensed data.

In this aspect, the method may be further adapted to, in response to detecting the event, cause an alert (e.g., an alert to the patient device and/or a Web portal) related to the upcoming LOC event.

In this aspect, the method may be adapted to communicate, to one or both of a patient or a caregiver, one or both of an expected severity of the LOC event or an expected initiation of the LOC event.

In this aspect, the method may be further adapted to communicate or inform about one or more of an expected severity of the LOC event, a severity of a past LOC event, a timing of when the LOC event is expected to occur, or a frequency of a plurality of LOC events, the method optionally adapted to communicate to one or more of the patient, a caregiver, a sponsor, a therapist, a clinician, or a nurse.

In this aspect, the method may be adapted to receive the input information from a wearable patient device.

In this aspect, the method may be further adapted to provide a correlation between one or more LOC events and one or more of a heart rate, a skin conductance, or patient glucose levels.

In this aspect, the method may be further adapted to provide guidance related to the effectiveness of medicine on one or more LOC events.

In this aspect, the method may be further adapted to provide a history of one or more of an event or a personalized event trigger (e.g. stress, sleep deprivation, alcohol drinking) that may optionally guide therapy in real-time or off-line.

In this aspect, the method may be further adapted to provide a nudge, warning, or guidance on suggested or needed biofeedback (such as without limitation meditation, breathing, etc.).

In this aspect, the method may be further adapted to facilitate physiologically-guided, as needed, on-demand therapy including real-time cognitive-behavioral therapy and health coaching during vulnerable moments.

In this aspect, the method may be further adapted to provide a risk forecast to the patient about their risk level of a LOC event based on stored patterns of physiology (including electrographic recordings, psychophysiology, and/or glucose measurements) behavior and logs.

In this aspect, the method may be further adapted to capture and record one or more LOC events and severity over a time history so patients and/or their caretakers/sponsors, clinicians and therapists can see the history of progress along with triggers for all events.

In this aspect, the method may be further adapted to cause or facilitate delivery of non-invasive electrical stimulation or ultrasound neuromodulation in an on-demand fashion to prevent or treat the LOC event.

One aspect of the disclosure is a patient device that is configured to alert or initiate an alert about of an upcoming LOC event.

In this aspect, the patient device may include stored thereon any of the computer executable methods (e.g., an App) herein.

In this aspect, the method (e.g., App) may be adapted to receive as input information indicative of sensed patient neural data, and detect an upcoming LOC event using at least the sensed data or information indicative of the sensed data. An App may be further adapted to, in response to detecting an event, cause an alert (e.g., an alert to the patient device and/or a Web portal) about the upcoming LOC event.

In this aspect, an App may be adapted to communicate or inform one or more of the following, to one or more of a patient, a caregiver, a sponsor, a therapist, a clinician, or a nurse: an expected severity of the LOC event, a severity of the LOC event, a timing of when the LOC event is expected to occur, or the frequency of a plurality of LOC events.

In this aspect, the patient device may be adapted to receive information from a wearable patient device, such as a device configured to receive patient neural signals.

### BRIEF DESCRIPTION OF THE DRAWINGS

FIG. 1 illustrates a merely exemplary and illustrative system which may include one or more of a personal device, a wearable device, or a web portal.

### DETAILED DESCRIPTION

The disclosure is generally related to LOC events.

One aspect of the disclosure is generally related to detecting a patient's upcoming, optionally imminent, LOC event and alerting the patient and/or caregiver about the detected and upcoming LOC event.

One aspect of the disclosure is related to computer executable methods (e.g., software, algorithms) that may be stored on one or more personal devices such as smartphones and computers, and are optionally stored as an Application (“App”).

One aspect of this disclosure is related to management of LOC conditions, and therapeutic approaches that can help and/or treat a patient susceptible to LOC events.

One aspect of this disclosure is related to LOC events, and correlating LOC events with other physiological parameters, such as without limitation, brain recordings, heart rate, glucose levels measured directly or indirectly, skin, conductance, etc.

One aspect of this disclosure is training LOC detection algorithms using more than one type of patient information (e.g., EMG data; EEG data (extracranial and intracranial); Glucose levels; Pupil size and eye tracking; heart rate variability; blood pressure; skin conductance) to better predict or detect one or more aspects of an upcoming LOC event.

LOC is expected to heavily relate to glucose levels. Preliminary evidence suggests that eating disorder behaviors have a substantial impact on blood glucose levels and glucose-related variables.

As used herein, computer executable methods may refer to one or more of software, algorithms, Apps, etc. In some preferred embodiments, an App is adapted and configured to receive data, or information indicative of data that is sensed or detected from a patient wearable device. In some embodiments the patient wearable device may be an implanted device that is adapted to sense physiological signals (e.g., neural signals), and communicate the signals or information indicative of the signals to the App (directly or indirectly), which may be stored on a personal device (e.g., smartphone, watch, etc.).

One aspect of the disclosure is a system that includes an App that is adapted to be stored on a personal device such as an iOS-operating or Android phone, and additionally a secure web portal. The App and the Web Portal may be configured such that Summary data on the LOC events related to any undesirable impulse can be accessed and viewed on both the App and the Web portal. The App may be configured to gather data, optionally via Bluetooth, from the wearable device and/or system, execute one or more proprietary algorithms on the data to determine if an LOC event is imminent or otherwise upcoming in the near future, and then initiate or cause to be initiated a notification alert to the patient when an LOC event is imminent. In addition, the App may be configured to cause biofeedback for the patient to be displayed on a display of the personal device (e.g., smartphone, watch) in response to a detected alert.

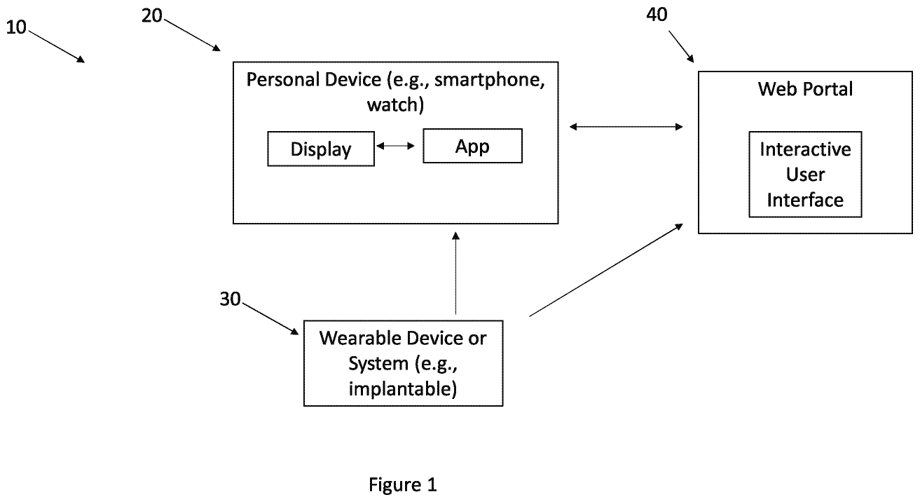

FIG. 1 illustrates a merely exemplary and illustrative system 10, which in this example includes a personal device 20, wearable device 30, and optional web portal 40. Arrows between the system components illustrate exemplary communication, which may be one or two-way communication. Any of the arrows may alternatively be one or two-way. The communication may be wireless communication (e.g., Bluetooth), while in some systems one or more communications may be wired.

The wearable device(s) may comprise a variety of devices, and may include a device that is adapted to be implanted within or on a patient's brain, and may be adapted to sense neural signals therefrom from which a LOC event can be detected. By way of example only, U.S. Pat. App. Pub. No 2016/0250476 illustrates a merely exemplary device that may be considered a wearable device that may be used with the systems herein (e.g, personal devices and web portals).

The systems herein, including one or more components thereof, may be configured to provide one or more of the following functionalities. It is understood that the systems herein need not necessarily provide all of these functions. Additionally, any of the functionality may be provided by one or more components of any of the systems herein, alone or together. For example, users of the systems herein need their personal data to be securely transferred, protecting personal health information from any potential breach. The systems herein are preferably adapted such that users can view the following LOC metrics via web and app interfaces (e.g., Web portal and/or App): Patient LOC diaries (timestamp log/history and severity rating for tracking in post-alert reports/surveys); psychophysiology reports (date/time of alerts given to the patient; weight logs; lifestyle logs. Systems herein are preferably adapted such that patients can give their clinicians and caretakers access to their LOC data. Systems herein are preferably adapted such that patients can wirelessly synch their wearable devices to the App (e.g., via Bluetooth). Systems herein are preferably adapted such that patients can wirelessly synch their App to a cloud-based web portal (e.g., via Bluetooth). Systems herein are preferably adapted such that the patient is alerted via the App interface when a LOC event is about to occur. Systems herein are optionally adapted such that patients are given biofeedback (e.g., breathing exercise, meditation, etc.) on the App interface, optionally before they lose control or before the LOC event. Systems herein are preferably adapted such that patients are able to log and rate the severity of the LOC events. Systems herein are also preferably and optionally adapted such that patients are able to log other life events that may have caused stress or anxiety (“lifestyle logs”). Systems herein are preferably adapted such that patient is able to capture their weight via a Bluetooth scale that is configured to communicate with and/or synchronize with the App. Systems herein are preferably adapted such that caregivers are alerted via an App when an LOC event occurs, which may include one or more set thresholds. Systems herein are preferably but optionally adapted such that the App signal pairs to App-based or weblink-based real-time, on-demand, mental health coaching or therapy if needed. This may be via a separate App, optionally marketed and commercialized by a different business entity as the Apps discussed herein. Systems herein are preferably but optionally adapted such that one or more computer executable methods (e.g., algorithms) are configured to be trained on one more LOC alerts and/or the feedback provided by the patient/caregiver on the LOC alerts and their accuracy. Systems herein are preferably but optionally adapted such that the App is adapted to automatically check for downloadable software updates that can ensure each user has the most up to date capabilities. Systems herein are preferably but optionally adapted such that caregivers can access and view historical data obtained (optionally stored) by the App on the web portal through a secure log-in (e.g., see exemplary communication between personal device 20 and web portal 40 in FIG. 1).

The systems herein, including one or more components thereof, may be configured to provide one or more of the following functionalities. It is understood that the systems herein need not necessarily provide all of the functions that follow. Additionally, any of the functionality may be provided by one or more components of any of the systems herein.

### Access:

Systems herein are preferably adapted such that a web portal and an App are accessible and viewable on any commercially available online web browser, preferably require a secure log-in, are preferably HIPAA compliant, and individuals must be granted permission to view a patient's data on the web portal and App.

### Web Portal View—Clinicians/Patients/Caretakers (or Other Third Party Caregiver):

Systems herein are preferably adapted such that the Web Portal is configured to display one or more of the following: patient logged events; automatically detected events; psychophysiological events logged by device(s) (e.g., Scalp; Wearable); weight log; patient alert log (date/time of all alerts given to patient); log of patient response to alerts to examine and confirm accuracy/relevance; lifestyle logs; and a diary for general or behaviorally relevant notes for specifics.

### App—Patient Use:

Systems herein are preferably adapted such that one or more of the following items can be entered on the App: Loss of Control event (e.g., initiation thereof); LOC rating or severity; Food log; Lifestyle log; Body weight.

Systems herein are optionally adapted such that the patient's weight can be collected wirelessly (via Bluetooth) using a commercially available wireless scale and synchronized with the App and/or Web Portal.

Systems herein are optionally adapted such that one or more of following data (or information in indicative thereof) can be processed using one or more proprietary algorithms to detect an LOC event and display one or more alerts to the patient of a possible LOC event: Intracranial and scalp electrographic data (change is frequency power from baseline and eventually connectivity); functional near-infrared spectroscopy (fNIRS), and magnetoencephalography (MEG); EMG data; Glucose levels; Pupil size and eye tracking; Heart rate increase and/or heart rate variability decrease; skin conductance increase.

Systems herein are preferably but optionally adapted such that, in response to a LOC alert, the system may be adapted to display biofeedback notifications to the patient (e.g., breathing, meditation, etc.).

### App—Patient/Caretaker/Clinician View:

Systems herein are preferably but optionally adapted such that one or of the following data, also available on the Web Portal view, will be available on the App interface: patient logged events; psychophysiological events logged by wearables (e.g., scalp, wearable); weight log; patient alert log (date/time of all alerts given to patient); lifestyle log.

### App—Caretaker Alerts:

Systems herein are preferably adapted such that thresholds may be set for caretakers to be alerted via the App when LOC events have occurred.

The systems and methods herein may also be adapted to perform one or more of the following functions or provide one or more of the following features: examine via correlation analyses how the LOC events correlate to other physiologic variables such as heart rate, skin conductance, etc; guide how effective other approaches like medicine are on LOC events and related; provide an electrographic diary of events and personalized event triggers (e.g. stress, sleep deprivation, alcohol drinking) that can guide therapy in real-time or off-line; provide guidance on need for biofeedback (e.g., meditation, breathing, etc.); provide physiologically-guided, as needed, on-demand warnings or nudges or therapy including real-time cognitive-behavioral therapy and health coaching during vulnerable moments; provide a risk forecast to patients about their risk level of an LOC event based on stored patterns of behavior and logs; the LOC events and severity will be captured over time so patients and their caretakers/sponsors, clinicians and therapists can see a full history of progress along with triggers for the LOC events; and/or deliver non-invasive electrical stimulation or ultrasound neuromodulation in an on-demand fashion as needed.

Even if not specifically indicated, one or more techniques described in this disclosure may be implemented, at least in part, in hardware, software, firmware or any combination thereof. For example, various aspects of the techniques or components may be implemented within one or more processors, including one or more microprocessors, digital signal processors (DSPs), application specific integrated circuits (ASK's), field programmable gate arrays (FPGAs), programmable logic circuitry, or the like, either alone or in any suitable combination. The term “processor” or“processing circuitry” may generally refer to any of the foregoing circuitry, alone or in combination with other circuitry, or any other equivalent circuitry.

Such hardware, software, or firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read only memory (ROM), non-volatile RAM (NVRAM), electrically erasable programmable ROM (EEPROM), Flash memory, and the like. The instructions may be executed by a processor to support one or more aspects of the functionality described in this disclosure.

What is claimed is:

1. A computer executable method stored on a device, the method adapted to initiate an alert of an upcoming patient Loss of Control (“LOC”) event.

2. The method of claim 1, wherein the method is adapted to receive as input sensed patient neural data or information that is indicative of sensed patient neural data, and detect an upcoming LOC event using at least the sensed data or the information indicative of the sensed data.

3. The method of claim 2, wherein the method is further adapted to, in response to detecting the event, cause the alert (e.g., an alert to the patient device and/or a Web portal) related to the upcoming LOC event.

4. The method of claim 3 wherein the method is adapted to communicate, to one or both of a patient or a caregiver, one or both of an expected severity of the LOC event or an expected initiation of the LOC event.

5. The method of claim 4, wherein the method is further adapted to communicate or inform about one or more of an expected severity of the LOC event, a severity of a past LOC event, a timing of when the LOC event is expected to occur, or a frequency of a plurality of LOC events, the method adapted to communicate to one or more of the patient, a caregiver, a sponsor, a therapist, a clinician, or a nurse.

6. The method of claim 2, wherein the method is adapted to receive the input information from a wearable patient device.

7. The method of claim 2, wherein the method is further adapted to provide a correlation between one or more LOC events and one or more of a heart rate, a skin conductance, or patient glucose levels.

8. The method of claim 2, wherein the method is further adapted to provide guidance related to the effectiveness of medicine on one or more LOC events.

9. The method of claim 2, wherein the method is further adapted to provide a history of one or more of an event or a personalized event trigger (e.g. stress, sleep deprivation, alcohol drinking) that may optionally guide therapy in real-time or off-line.

10. The method of claim 2, wherein the method is further adapted to provide a nudge, warning, or guidance on suggested or needed biofeedback (such as without limitation meditation, breathing, etc.).

11. The method of claim 2, wherein the method is adapted to facilitate physiologically-guided, as needed, on-demand therapy including real-time cognitive-behavioral therapy and health coaching during vulnerable moments.

12. The method of claim 2, wherein the method is further adapted to provide a risk forecast to the patient about their risk level of a LOC event based on stored patterns of physiology (including electrographic recordings, psychophysiology, and/or glucose measurements) behavior and logs.

13. The method of claim 2, wherein the method is further adapted to capture and record one or more LOC events and severity over a time history so patients and/or their caretakers/sponsors, clinicians and therapists can see the history of progress along with triggers for all events.

14. The method of claim 2, the method further adapted to cause or facilitate delivery of non-invasive electrical stimulation or ultrasound neuromodulation in an on-demand fashion to prevent or treat the LOC event.

15. A patient device that is configured to alert or initiate an alert about of an upcoming LOC event.

16. A device of claim 15, wherein the patient device includes stored thereon any of the computer executable methods (e.g., an App) from any one of claims 1-14.

17. The device of claim 16, where the App is adapted to receive as input information indicative of sensed patient neural data, and detect an upcoming LOC event using at least the sensed data or information indicative of the sensed data.

18. The device of claim 17, wherein the App is further adapted to, in response to detecting the event, cause an alert (e.g., an alert to the patient device and/or a Web portal) about the upcoming LOC event.

19. The device of claim 18, wherein the App is adapted to communicate or inform one or more of the following, to one or more of a patient, a caregiver, a sponsor, a therapist, a clinician, or a nurse: an expected severity of the LOC event, a severity of the LOC event, a timing of when the LOC event is expected to occur, or the frequency of a plurality of LOC events.

20. The device of claim 15, wherein the patient device is adapted to receive information from a wearable patient device, such as a device configured to receive patient neural signals.
